(19) United States
(12) Patent Application Publication
(10) Pub. No.: US 2012/0277857 A1
(43) Pub. Date: Nov. 1, 2012

(54) Intraocular Lens with Fresnel Prism

(75) Inventors: Daniel Purchase, Sussex (GB); Peter Toop, Sussex (GB)

(73) Assignee: RAYNER INTRAOCULAR LENSES LIMITED, Buckinghamshire (GB)

(21) Appl. No.: 13/511,529

(22) PCT Filed: Nov. 23, 2010
(86) PCT No.: PCT/GB10/51944

§ 371 Date: May 23, 2012

(30) Foreign Application Priority Data
Nov. 23, 2009 (GB) 0920505.5

(51) Int. Cl. A61F 2/16

(52) U.S. Cl. 623/6.26

(57) ABSTRACT

An intraocular lens is described which comprises, as one face thereof, a linear Fresnel prism array with facets angled relative to the optical axis of the lens so as to deviate light incident thereon to an off-axis position. The facets are modified so as to reduce at least one of diffraction effects and astigmatism associated with the Fresnel prism. In particular, by varying the pitch of the prism elements across the array, which may comprise varying their size, a diffraction grating effect can be reduced or negated, such that light is not diffracted into undesirable orders and multiple images can be avoided. Furthermore, chromatic angular dispersion associated with the diffraction grating effect may be reduced. The pitch variation can be random. By varying the angle of the facets across the array, astigmatism that would otherwise result from the presence of the Fresnel prism can also be compensated.

### FIELD OF THE INVENTION

The invention relates to an intraocular lens (IOL), and in particular to an improved IOL with Fresnel prism that can be used to reduce the effects of age-related macular degeneration (ARMD).

### BACKGROUND TO THE INVENTION

The treatment of focal macular diseases, and in particular ARMD, represents a major problem. Since the intact macula provides the vision that is required for reading, driving etc (but not for peripheral vision), the fact that there is no effective treatment for its degeneration means that many people increasingly retain peripheral vision only.

In order to solve this problem, it has been proposed that the retina should be surgically repositioned in the eye. A more practical solution is to optically deviate the image of the fixation point from the macula to a point on the retina where there are healthy cells. Although these cells may not function as well as the macular cells, an adequate degree of vision may be retained.

Among other things, this is proposed in U.S. Pat. No. 6,197,057. In particular, each of FIGS. 25, 27, 31 and 33 of U.S. Pat. No. 6,197,057 discloses a supplemental ions, i.e. an intraocular lens that is provided in addition to the natural, crystalline lens or to a biconvex IOL. All these drawings show a supplemental lens that is a conventional prism. The consequence is that the image is moved, away from the macula. Elsewhere in the specification, it is suggested that a Fresnel lens should be used as the supplemental IOL (column 9 line 13), and also that the lens should be “Fresnel-shaped”, again in the context of a supplemental lens). It is unclear what form the “Fresnel-shaped” lens should take.

WO03/047466 discloses an IOL that comprises a Fresnel prism. In this way, the focusing power of the IOL can be provided by a conventional lens that is modified so that light is focused on a (healthy) part of the retina that is not the macula. Such an IOL can be used to alleviate the effects of ARMD.

However, although a lens of the type disclosed in WO03/047466 provides a compact means to achieve the desired deviation of light, it can give rise to some undesirable optical effects, including optical aberrations. Thus, there is a need for an improved IOL having the benefits of the Fresnel prism type lens, but without the disadvantages.

### SUMMARY OF THE INVENTION

According to a first aspect of the present invention, there is provided an intraocular lens having an optical axis, the lens comprising, as one face thereof, a Fresnel prism comprising an array of elongate prism elements which are parallel to one another along their length, each prism element having an elongate facet which is oriented such that a perpendicular to the facet is at an angle to the optical axis,
wherein the array of prism elements is configured to deviate light incident thereon to an off-axis position lying in a plane defined by the optical axis and the perpendicular to any of the angled facets,
and wherein one or more of the pitch and the size of prism elements is non-uniform across the array and is selected to reduce a diffraction grating effect associated with the array of prism elements, whereby light incident on the lens is preferentially directed into the zero order diffraction direction and chromatic angular dispersion is reduced.

This aspect of the invention arises from the observation that a lens of the type disclosed in WO03/047466 has an undesirable optical diffraction grating type effect due to the periodic nature of the prism spacing in a typical Fresnel prism. A solution to this problem, according to the present invention, is an intraocular lens comprising, as one face thereof, a linear Fresnel prism array whose facets have been modified to reduce this diffraction effect. In particular, by varying the pitch, which may comprise varying the size of the prism elements, the diffraction grating effect can be reduced or negated, such that light is not diffracted into undesirable orders and multiple images can be avoided. Furthermore, chromatic angular dispersion associated with the diffraction grating effect may be reduced.

It should be noted that the Fresnel prism in the lens of the present invention does not constitute a Fresnel lens or zone-plate, and there is no circular symmetry to the array of prism elements itself, although other aspects of the lens may have circular symmetry The Fresnel prism in the present invention is a linear array of elongate prism elements located at one surface of a lens, which is intended to deviate light passing through the lens. In other regards the lens may be more conventional in construction, although various constructions are possible.

In a preferred embodiment, one or more of the pitch and the size of prism elements in the array has been randomised to reduce the diffraction grating effect. A random variation in the prism size, and therefore prism pitch, can avoid the constructive interference effect which would otherwise lead to light energy being directed into diffraction orders other than the desired zero order.

The randomisation may be similar across the array or else may be different one region as compared to another, for example in a region of the array proximate the optical axis as compared to a region distal the optical axis. In any case, it is desirable to ensure the presence of randomisation the region proximate the optical axis as well as across the whole array.

Preferably, the pitch of the prism elements in the array is in the range 50 μm to 500 μm, with the variation or randomisation of the localised pitch or spacing of the prism elements resulting in the pitch lying within this range.

In some embodiments, it is preferred that the pitch of the prism elements in the array varies by an amount in the range 0 μm to 50 μm. It should be noted that this is the variation in pitch, not the absolute value of the pitch. In other embodiments, it is preferred that the pitch of the prism elements in the array varies by an amount in the range 0 μm to 130 μm. A larger variation can more effectively reduce the diffraction grating effect and is desirable, providing the corresponding size of the prism elements is compatible with a given application and fabrication technique.

Without wishing to be bound by theory, when a prism is used in a converging light beam, it adds optical aberrations to the beam (astigmatism and coma). This is true for a single prism and for a Fresnel prism array. The astigmatism results in a separation of the sagittal and tangential foci of the converging rays. Therefore, rays in the plane of deviation now come to a focus closer to the IOL than those in the orthogonal plane. It is therefore also desirable to compensate for this astigmatism.

Therefore, in some embodiments of the invention it is preferred that a facet angle of prism elements is nonuniform across the array and is selected to compensate for astigmatism that would otherwise result from the presence of the Fresnel prism. The prism angle can be varied across the diameter of the lens, which can prevent the prism focusing power addition that occurs in converging light. Varying the angle can also have an additional effect. If each of the individual prisms has a very slightly different angle, tuned depending on the predicted angle of the ray that will hit it, it may be possible to ensure that all the rays exiting each prism surface converge at a single point, thereby correcting astigmatism.

It should be noted that, although the variation or tuning of the prism facet angle has been discussed in the context of the first aspect of the invention, this feature may have independent utility in the context of an IOL comprising a Fresnel prism.

According to a second aspect of the present invention, there is provided an intraocular lens having an optical axis, the lens comprising, as one face thereof, a Fresnel prism comprising an array of elongate prism elements which are parallel to ore another along their length, each prism element having an elongate facet which is oriented such that a perpendicular to the facet is at an angle to the optical axis,
wherein the array of prism elements is configured to deviate light incident thereon to an off-axis position lying in a plane defined by the optical axis and the perpendicular to any of the angled facets,
and wherein the angle of the prism element facets is non-uniform across the array and is selected to compensate for astigmatism that would otherwise result from the presence of the Fresnel prism.

Preferably, the facet angles vary monotonically across at least a portion of the array to compensate for the astigmatism.

In one particular embodiment, the angle of the facets is in the range 37.5 to 38.5 degrees, although any other suitable angle or range of angles may be used according to the specific application. The mean facet angle will generally be determined by the angular deviation that the Fresnel prism is required to provide when implanted in a patient's eye. This, in turn, will be determined by selection of a point on the retina where there are healthy cells and to which the image of the fixation point is to be deviated from the macula. The variation in facet angle, including the range of variation, will largely be determined by the requirement to compensate for the astigmatism that would otherwise result from the presence of the Fresnel prism.

In a further preferred embodiment, an intraocular lens of the invention comprises also a toric lens surface. This may correct the prism power addition. By pre-calculating the additional focusing power added by the rear prism surface in one axis, the optical front surface can be made with the correct optical power in both axes, that is to say a toric surface with less optical power in the axis of beam deviation. The toric lens surface can be used in combination with either or both of the first and second aspects of the invention.

The prism elements may be formed on a planar surface. Alternatively, the prism elements may be formed on a non-planar or curved surface.

The Fresnel prism component itself may have any of a variety of suitable designs. These include planar (flat disc), cylindrical (curved disc) and spherical (meniscus disc).

Preferably, in an IOL of the invention, the Fresnel prism is on the anterior surface, when in use. In this embodiment, the focus power addition is not so great, since the prism surface is in a less convergent beam.

The lens may be used in the eye, in either orientation, but it is generally preferred that a smooth face should face the posterior capsule. That face of the lens having the Fresnel prism may be made smooth, by covering it with a translucent material.

A lens used in this invention may be of conventional size and may be made of any suitable material. General characteristics of such lenses are known. The lens may he made of a rigid or foldable material. Suitable materials are those used for intraocular lenses and include both hydrophobic and hydrophilic polymers containing acrylate and methacrylate such as polymethyl methacrylate, and silicone elastomers such as dimethylsiloxane.

If necessary or desired, a lens of the invention may include one, two or more haptics. As is known, they may be attached to the body of the lens at its perimeter, and may extend radially or tangentially.

A lens used in this invention will usually have only one power. A range of lenses may be produced, each having a different power. Alternatively, the inclusion of a supplementary lens may be used to achieve the correct dioptric power for each eye.

According to a third aspect of the present invention, there is provided a combination of an intraocular lens according to according to the first or second aspect, and a second intraocular lens.

Preferably, the second lens has a toric shape to compensate for astigmatism in the lens combination.

According to a fourth aspect of the present invention, there is provided a method for the treatment of a macular condition requiring a change of focused image position, which comprises replacing a patient's crystalline lens by a lens according to the first or second aspects of the invention or a lens combination according to the third aspect of the invention.

According to a fifth aspect of the present invention, there is provided a method for the treatment of a macular condition requiring a change of focused image position, which comprises implanting into a patient's eye a lens according to the first or second aspects of the invention or a lens combination according to the third aspect of the invention in order to supplement the patient's crystalline lens or an existing intraocular lens or lens combination.

The methods of the fourth and fifth aspect of the invention are particularly applicable where the macular condition is age-related macular degeneration.

A lens of the invention may be used, following removal of the crystalline lens, for the treatment of any macular condition requiring a change of focused image position on the retina. The lens is particularly useful for treatment of ARMD. Its function may be visualised by substituting such a lens for the crystalline lens/IOL plus supplementary lens shown in FIGS. 25, 27, 31 and 33 of U.S. Pat. No. 6,197,057.

As will be appreciated by those skilled in the art, the present invention provides for a much improved design of IOL based on a Fresnel prism, and which addresses a number of problems that may arise in known Fresnel prism intra-ocular lenses. Moreover, optimised design of the prism elements in the Fresnel prism array, together with careful design of other lens surfaces, allow a high performance lens to be customised for implantation in a patient's eye.

### BRIEF DESCRIPTION OF THE DRAWINGS

Examples of the present invention will now be described in detail with reference to the accompanying drawings, in which;

FIG. 1 is a schematic cross-sectional view of an IOL comprising a Fresnel prism;

FIGS. 2 A and 2B show, respectively, a side view and top view schematic illustration of a lens arrangement in the eye showing the optical aberration caused by an IOL as shown in FIG. 1;

FIGS. 3A and 3B show, respectively, a side view and top view schematic illustration of a lens arrangement in the eye, including a Fresnel prism IOL according to the invention;

FIG. 4 is a schematic of the optical bench system used to simulate an eye containing an IOL and test the optical lens performance;

FIGS. 5A and 5B show CCD images of a test target obtained using the system shown in FIG. 3 where the IOL was, respectively, a PMMA 26.5 D standard spherical lens and 22 D lens of the present invention;

FIGS. 6A and 6B show images illustrating the result of limiting the range of wavelengths passing through the lens to about 10 nm using a band-pass optical filter. In FIG. 6B the test target was illuminated with a laser spot in addition to background room lighting;

FIG. 7A illustrates interference between wave fronts originating from two point sources, indicating the angles for constructive interference;

FIG. 7B shows an example of the intensity profile across a screen in the arrangement of FIG. 7A;

FIG. 8A shows the calculated interference pattern in angular space for 100 emitters regularly spaced at 51 microns, at wavelength 546 nm, assuming uniform diffraction efficiency;

FIG. 8B shows the calculated interference pattern of FIG. 8A with an estimated diffraction efficiency curve applied to the data;

FIG. 9A shows the calculated interference intensity profile corresponding to that of FIG. 8A but with emitter spacing randomised by up to +20 microns (i.e. 51 to 71 microns);

FIG. 9B shows the calculated interference intensity profile corresponding to that of FIG. 8A but with emitter spacing randomised by up to +50 microns (i.e. 51 to 101 microns);

FIGS. 10A and 10B show, respectively, a plan view and side view of a Fresnel prism lens in accordance with the present invention;

FIGS. 10C and 10D show an expanded portion of the Fresnel prism lens of FIG. 10B, respectively, with uniform prism height and pitch and with varying prism height and pitch (spacing Xₙ as given in Table 2);

FIG. 11 shows a CCD image of a test target using the system shown in FIG. 4 where the IOL used a random prism spacing 22 D lens, with prism spacing Xₙ as given in Table 2 (the image also includes laser pointer spot);

FIG. 12A shows the calculated interference intensity profile for any array of prisms with spacing randomised in the range 130 micron to 260 micron;

FIG. 12B shows the shows central 3 mm from FIG. 12A, highlighting the significant intensity of the closest side lobes (up to ˜50%);

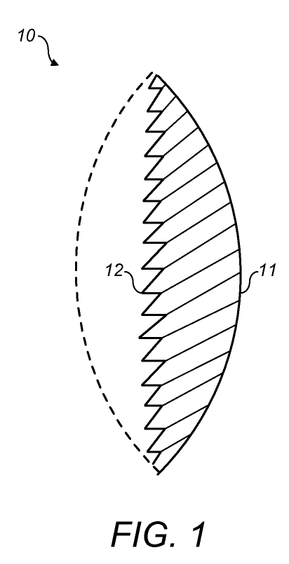

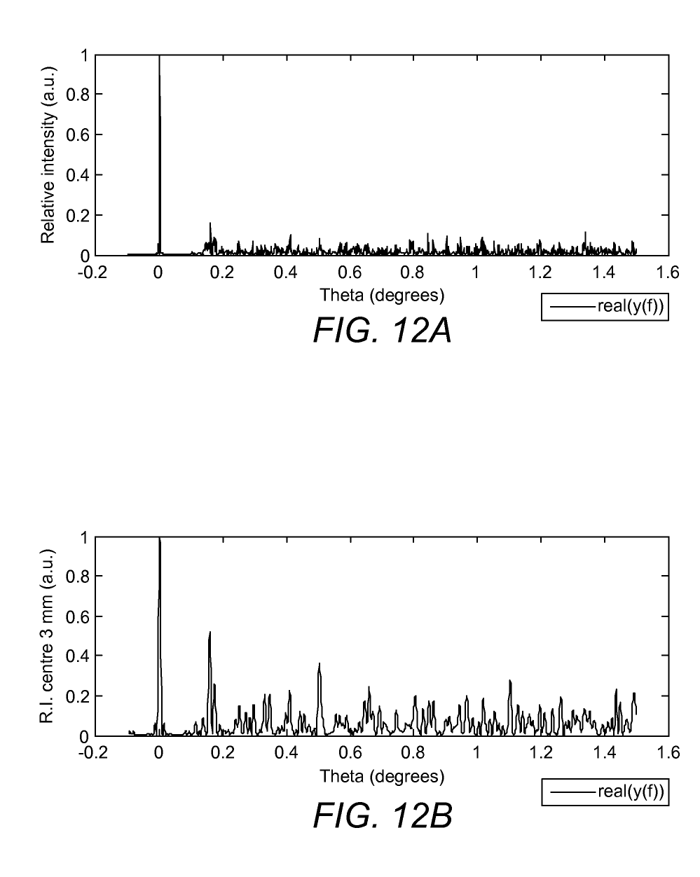

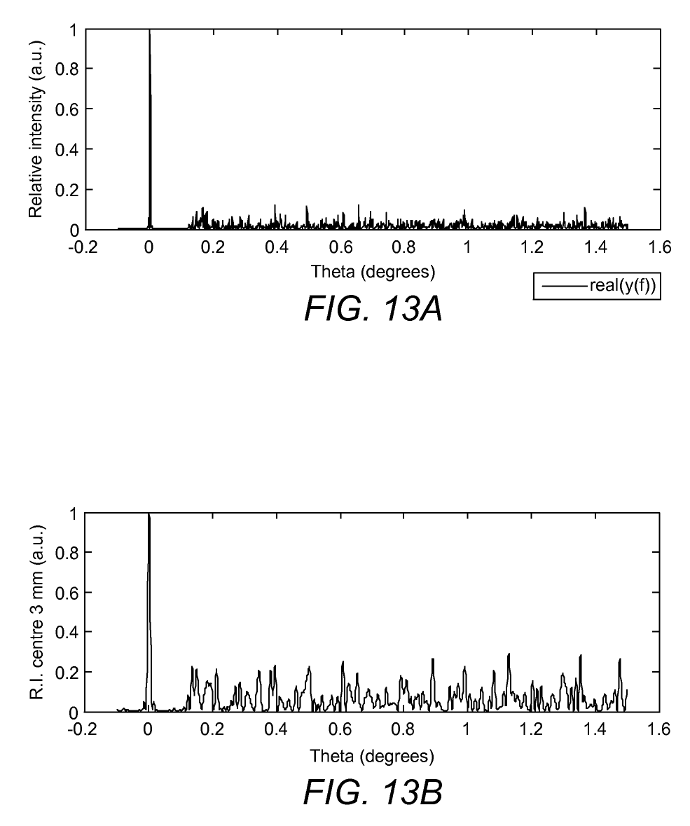

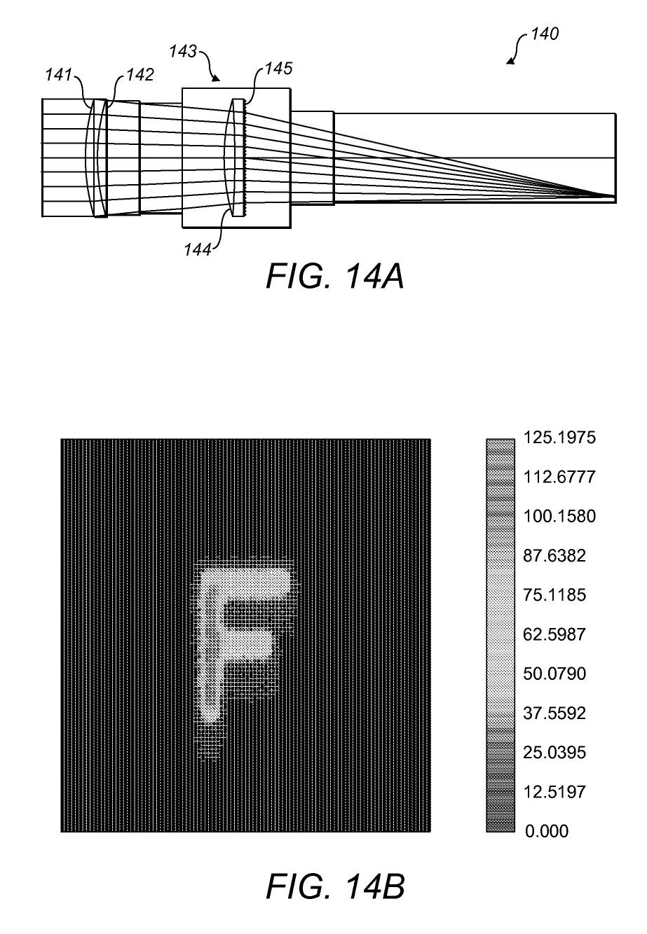

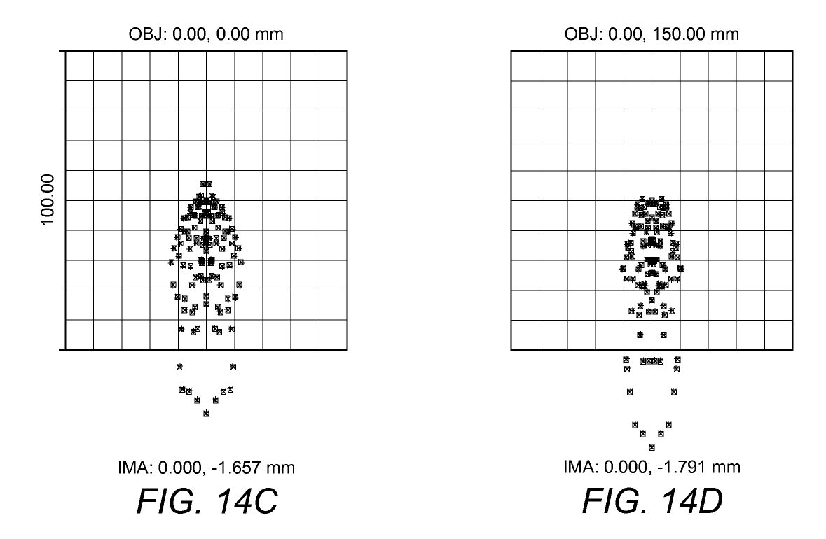

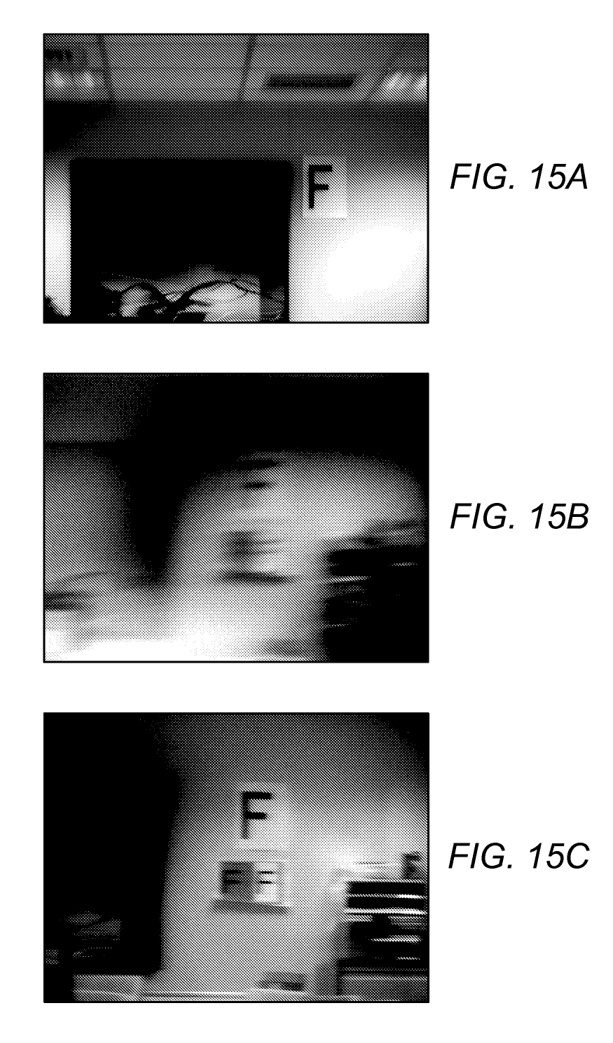

FIGS. 1 3A and 13B show the results of a similar calculation to those of FIGS. 12A and 12B, but with greater randomisation in the central 3 mm and highlighting the comparative lack of noticeable side lobe structure;

FIG. 14A illustrates ray tracing through a simulated eye with a 21 D IOL according to the present invention, having a random prism spacing in the range 130 μm to 260 μm according to Table 3, and an anterior toric surface;

FIG. 14B shows the image quality of a letter “F” imaged through the system shown in FIG. 14A;

FIGS. 14C and 14D show a spot diagram for the ray traced system of FIG. 14A; and,

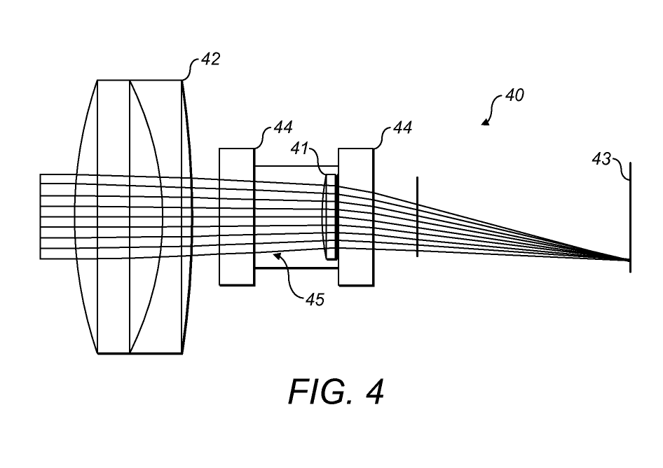

FIGS. 15A, 15B and 15C show CCD images of a test target obtained using the system shown in FIG. 4 where the IOL used was, respectively, a PMMA 26.5 D standard spherical lens, a 21 D Fresnel prism lens with machined regular spacing, and a 21 D Fresnel prism lens with machined random spacing and toric anterior surface according to the present invention.

### DETAILED DESCRIPTION

The invention will now be illustrated by way of example only with reference to the accompanying drawings. FIG. 1 comprises what is essentially one-half of a conventional lens 10, having a curved surface 11, and an opposed surface 12 in the form of a Fresnel prism. The Fresnel prism is essentially a linear array of prism elements having a constant profile in one direction and a modulated profile in the orthogonal direction. As shown in FIG. 1, the modulation of the Fresnel prism surface can take the form of a sawtooth, with each prism element having one facet that is essentially parallel to the optical axis of the lens and one facet that is angled with respect to the optical axis.

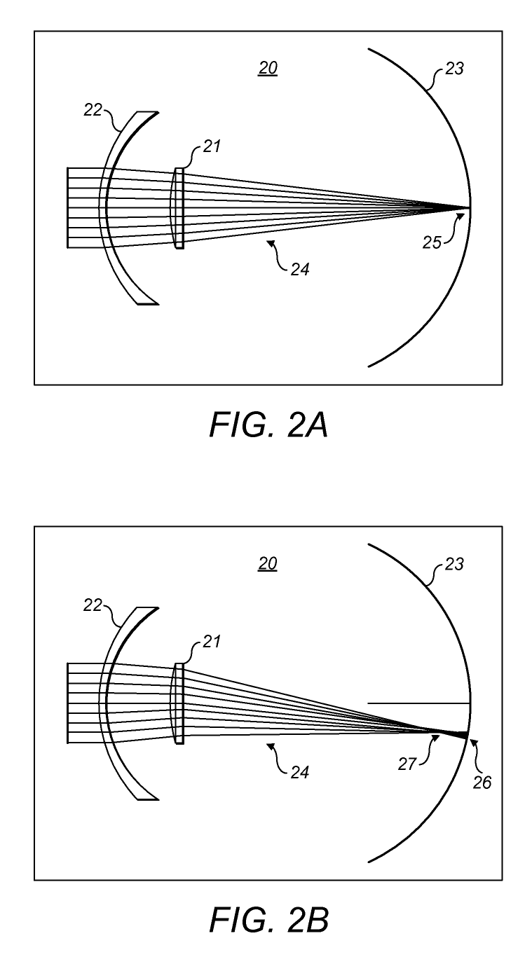

FIGS. 2A and 2B shows optical rays 24 traced through a Fresnel prism intraocular lens 21 of the type shown in FIG. 1 placed in a schematic eye 20, and illustrate an optical aberration caused by the prismatic intraocular lens. The IOL shown comprises a spherical lens surface (the surface facing the cornea 22 of the eye) and a Fresnel linear prism array (the surface facing the retina 23). The angled facets of the prism elements in the array are configured to deviate light incident thereon to an off-axis position lying in a plane defined by the optical axis and a line perpendicular to the angled facets. Thus, light rays incident on the lens in this plane will be so deviated, whilst light rays incident on the lens in a plane orthogonal to this will not be.

FIG. 2A shows the latter situation, with light rays focussing to an undeviated point of the retina 25 and also on the optical axis. By contrast, FIG. 2B shows the former situation, where light rays are deviated towards an off-axis point on the retina 26. Moreover, due to astigmatism introduced by the Fresnel prism, light rays in this plane actually converge to a point 27 not lying on the retina. As shown in FIG. 2B, the rays are focussed short of the retina, thereby leading to astigmatic aberration and a lack of sharpness in the image perceived by the eye. This is as a result of different focal lengths for orthogonal directions, with a shorter focal length (higher dioptre power) in the plane of image deviation.

It should be noted that., if the intraocular lens surfaces are exchanged, one for the other, a similar aberration will occur. Moreover, it should be noted that if the IOL were rotated in the eye, then the two planes defined above would also be rotated by the same amount. Thus, orientation of the lens determines the direction in which light is deviated by the Fresnel prism, and this can he selected in accordance with an off-axis point on the retina, which has been predetermined as suitable in view of the patient ARMD.

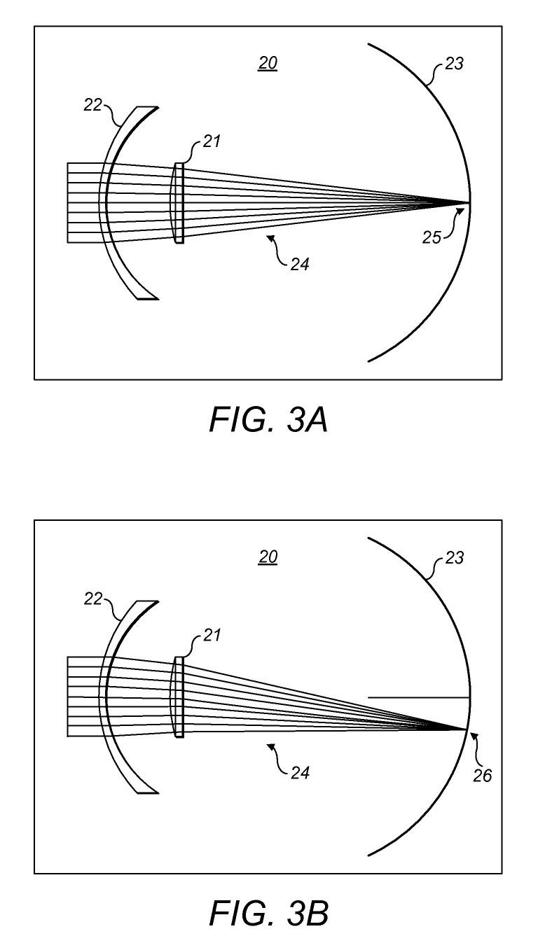

FIGS. 3A and 3B shows corresponding rays to those of FIGS. 2A and 2B traced through a schematic eye, but in which the astigmatism has been corrected or compensated for. This may be achieved using a prismatic intraocular lens according to present invention and, in particular, the second aspect of the invention, whereby the front optical surface and/or the prism facets have been modified to correct the astigmatism. FIG. 3A essentially corresponds directly to FIG. 2A, whilst FIG. 3B corresponds to FIG. 2B where the astigmatism is corrected. As shown in FIG. 3B, the rays in the orthogonal plane now converge to a single deviated point 26 on the retina.

In addition to the problem of optical aberrations, there are also optical effects associated with the presence of an array of elements of a size end spacing on the order of the wavelength of light or less. Without wishing to be bound by theory, the lens shown in FIG. 1 has a regular spacing of prism elements the Fresnel prism surface. As such, the array of elements acts very much like a high blaze angle transmission diffraction grating.

The diffraction grating effect has two main effects on the image: a) chromatic angular dispersion due to the sensitivity of diffraction angle with wavelength: and b) multiple images from the different diffraction orders. The angular separation of each order is given by m.λ=n.d. sin θ, where m is diffraction order, λ is wavelength of light, n is the surrounding medium refractive index, d is the grating spacing, and θ is the angle of diffraction. It is therefore an object of the invention to remove or mitigate the diffraction grating effect, and thereby the image quality can be increased and multiple images avoided or reduced to an imperceptibly low level of intensity. There are a number of ways in which this may be achieved.

In order to simulate the performance of an IOL using the present invention, it was necessary to develop models of the Fresnel Prism lens for calculating the expected performance purposes and also an optical bench system for simulating the performance of an IOL in a patient eye, such that representative imaging tests could be performed. Such tools would allow the performance of a conventional Fresnel prism IOL to be analysed as a baseline measurement and then compared to the performance of an improved Fresnel prism IOL according to the invention.

A number of experimental techniques were employed to investigate the Fresnel prism IOL. A Nickon microscope was used for visual inspection of prism structure. Laser spot imaging (using a 532 nm laser) enabled experimental visualisation of diffraction effects to determine the level of diffraction with a Fresnel prism IOL. As will be described below, a model “eye” with imaging CCD camera allowed image formation to simulated and the quality assessed. Finally, the use of a band-pass filter (10 nm band-pass centred at 546 nm) allowed me range of wavelengths entering the simulated eye to be reduced considerably, thereby allowing both monochromatic and chromatic effects to be observed and isolated.

FIG. 4 shows an optical bench system that was developed to simulate an eye 40 containing an IOL 41. A lens 42 was designed to simulate the behaviour of the cornea, whilst a CCD camera 43 represented the retina. The Fresnel prism lens 41 was disposed within an optical cell 44 containing a saline solution 45. Using this system it was possible to develop tests and experiment with the possible causes of unexpected visual artefacts. It was also possible to obtain an image similar to that projected onto the patient's retina.

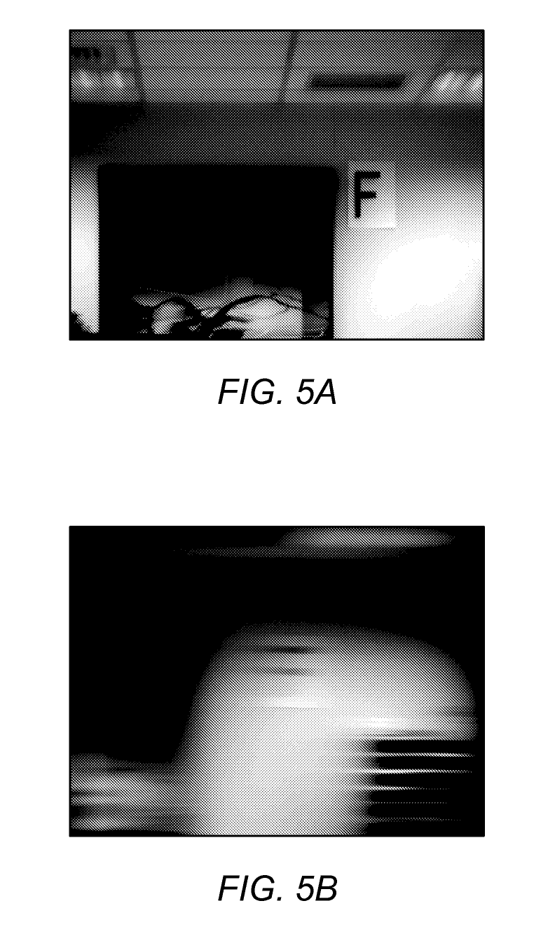

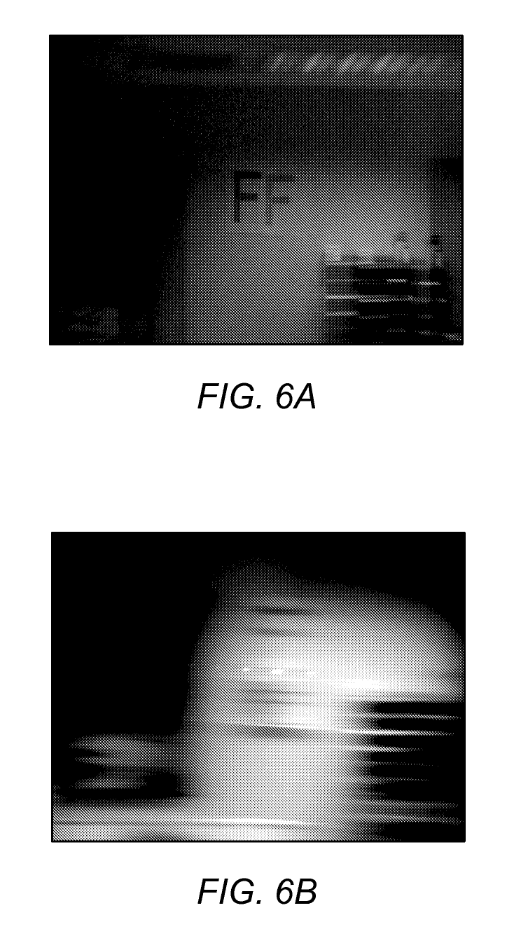

FIGS. 5A and 5B show images of a test target (a letter “F” approximately 250 mm high) recorded on the CCD camera at a distance of about 10 m, using an IOL of the type shown in FIG. 1 comprising a Fresnel prism having a uniform pitch. After investigation the cause of the poor imaging quality was discovered to be two fold, chromatic aberration caused by the dispersion of the prisms and diffraction caused by the close spacing and angle of the prism facets. By limiting the range of colours allowed through the system it was possible to test both the chromatic aberration and the diffraction introduced by the Fresnel prism IOL. The results are shown in FIGS. 6A and 6B. An additional test was carried out using a monochromatic light source (laser). This demonstrated the imaging quality of the lens minus any chromatic effects, but still illustrated any diffraction issues. FIG. 6A shows the images obtained under various test conditions.

It is clear from FIG. 6A that the imaging quality of the lens is acceptable, with the letter “F” and general background objects clearly visible. The double image is due to diffraction, an d this is confirmed in FIG. 6B, where the single illuminating laser spot is diffracted into multiple spots (just below the F) at the imaging plane of the CCD camera (patient's retina). Therefore, if the chromatic dispersion and diffraction can be controlled the optical performance of the lens will be perfectly acceptable for the intended purpose.

In practice, a certain level of diffraction could be tolerated, as the retina in a real human eye would simply ignore the additional image, if it is below a certain intensity when compared to the rest of the image on the retina. Moreover, before application to a real patient, additional information about the visual acuity of the retina as a function of distance from the visual axis would be required. In particular, an understanding of the patient condition in terms of the limit of the macular degeneration and whether the degeneration stable. Ideally, the Fresnel prism IOL would be designed for an image offset that is as small as possible to ensure the best visual acuity.

Very basic diffraction calculations (light and dark strips at the prism spacing) had suggested that the diffraction efficiency for a periodic Fresnel prism array would be very low, for example 10⁶ times less energy in the +1-diffraction order compared to the (zero) 0-order, and that therefore diffraction might not be a significant problem. However, as described above, initial experimental results showed that diffraction is occurring in a conventional Fresnel prism IOL and that there is significant energy in the diffracted light. In this regard, it was noted that a very important diffraction efficiency parameter for diffraction gratings is the grating blaze angle. In the Fresnel prism IOLs under test, the prism faces, or equivalently grating facets, are set at about 40 degrees. This high blaze angle will force energy into the higher diffraction orders, as was noted.

To back up the theory, simulations were performed using diffraction calculations, based on a coupled-wave model as formulated by M. G. Moharam, E. B. Grann, D. A. Pommet, and T. K. Gaylord in “Formulation for stable and efficient implementation of the rigorous coupled-wave analysis of binary gratings,” J. Opt. Soc. Am. A, vol. 12, pp. 1068-1076, May 1995, and by M. G. Moharam, E. B. Grann, D. A. Pommet, and T, K. Gaylord in “Stable implementation of the rigorous coupled-wave analysis of surface-relief gratings: enhanced transmittance matrix approach,” J. Opt. Soc. Am. A, vol. 12, pp. 1068-1076, May 1995.

The results of the calculation are shown in Table 1 in terms of the percentage of fight (diffraction efficiency) diffracted into a given order. As shown, taking an incident ray angle of 4 degrees (@633 nm), the diffraction efficiency was highest around −10 order.

TABLE 1

| Diffraction | |
|---|---|
| order | Efficiency % |
| −12 | 4 |
| −11 | 45 |
| −10 | 30 |
| −9 | 4 |

It is also notable that there is a significant split of energy between two diffraction orders. As noted previously, the angular separation of each order is given by m.λ=n.d. sin θ, where m is diffraction order, λ is wavelength of light, n is the surrounding medium refractive index, d is the grating spacing, and θ is the angle of diffraction. This calculation appears to correspond well with laser (532 nm) spot images, as shown in FIG. 6B. The spots are spaced by 48 pixels, which with a CCD pixel size of 5.6 microns imply a diffracted order spacing of 0.27 mm. At a wavelength of 532 nm, the theoretical separation between the −10 order and the −11 order is 0.82 degrees. Based on a rough distance of 17 mm from the back of the lens to the CCD chip, the expected spacing is calculated to be 17*Sin 0.82=0.24 mm, which is very close to the measured value.

The diffraction efficiency was also sensitive to the incident angle of the rays hitting the prisms. When the lens is in the capsular bag, the prism surface will be exposed to a range of angles determined by the size of the pupil and the focal length of the lens (i.e. roughly the distance from prism surface to retina). This range of angles will spread the light out over a range of diffraction orders. Also the diffraction angle is very sensitive to wavelength. Therefore, even if the incident polychromatic light were to hit the prism surface at a single angle, the light would be chromatically separated at the retina. This results in a very blurred image on the retina, as shown in FIG. 5B. Therefore, a prism lens design was required that removed or reduced the diffractive effect. In accordance with the invention, it was proposed that random prism spacing should remove the combined diffractive effect of the evenly spaced prisms.

This investigation required several different designs for the prism surface to allow diffraction effects to be compared. The prism lenses were generally compression molded from PMMA, although the high cost of producing mold tools makes this process expensive for test sample volumes. One alternative method for producing linear structures on a lathe is to use a fly-cutter configuration (where the cutting tool is mounted on the lathe spindle and the work piece is attached to the bed).

Diffraction at the prism surface will produce multiple output beams. In the ideal case with zero diffraction efficiency there would only be a single output beam, and this beam would be deviated from the input beam by an angle determined by the prism angles and refractive indices of the optic and surrounding medium. As diffraction efficiency increases, there will be noticeable additional beams either side of the central non-diffracted beam and increasing amounts of energy will be present in the additional beams, as diffraction efficiency continues to increase.

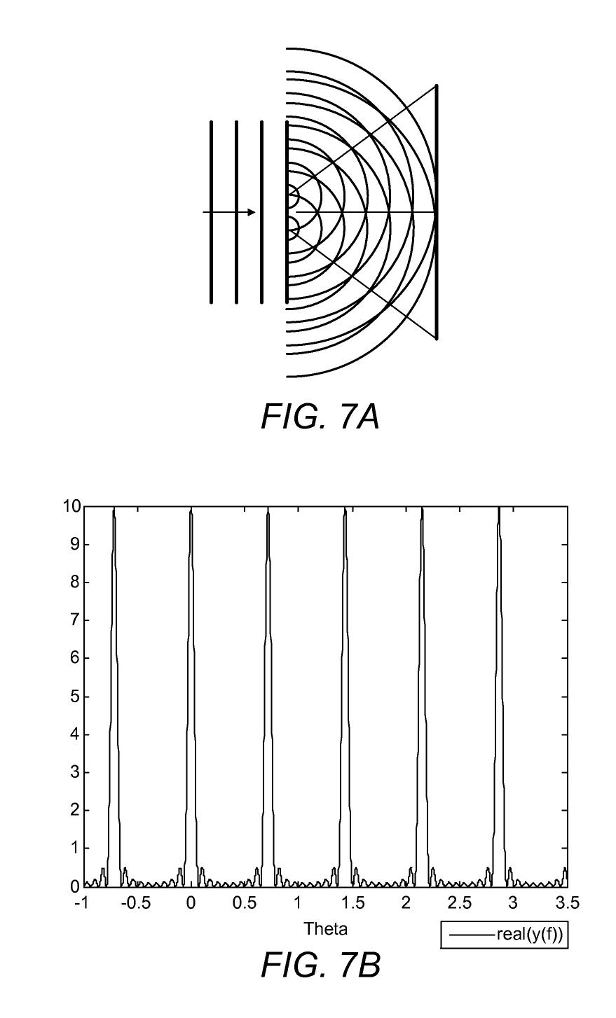

In order to investigate the behaviour of the Fresnel prism surface, a relatively simple model was adopted to simulate interference effects, in which the array of prism elements was modelled as a set of discrete spherical wave emitters, or point sources, each located at the centre of each prism face. This model does not incorporate diffraction theory but was been chosen as a simple and fast test model to investigate the effect of randomising the prism spacing on constructive interference of the point sources. FIG. 7A illustrates the underlying principles of this model, in two dimensions only, with two point sources, or emitters. If a screen were placed at the right hand side of the image then a series of light and fringes would be seen, as shown in FIG. 7B.

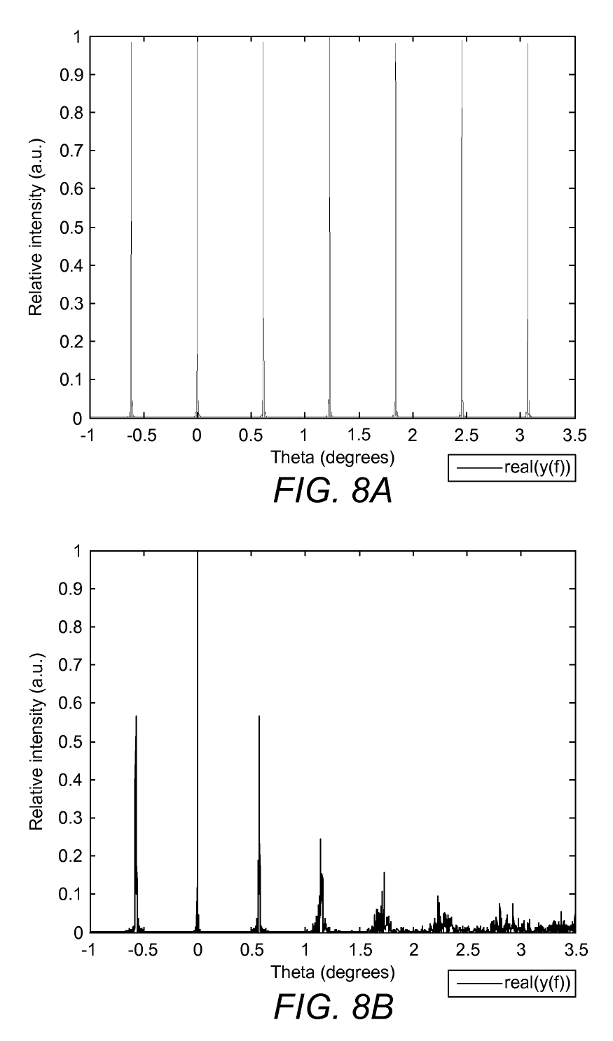

Using a design of Fresnel prism array with uniform prism size and spacing as a starting point, and placing a point source emitter at the centre of each prism face, we have 8 mm optic diameter and 0.04 mm prism depth with 38 degree prism face angle. This gives a prism spacing of 0.051 mm, calculated from 0.04/arctan(38°). This corresponds to about 118 prisms across the optic, calculated from 6 mm/0.051 mm. Therefore, the initial simulations were performed for a wavelength of 546 nm using 100 emitters in air, and spaced at 51 microns. The calculated angular intensity profile at some distance from the source plane is shown in FIG. 8A. In this calculation, detailed diffraction theory is not considered and the calculation is based purely on an interference model.

As can be seen in FIG. 8A, there is a regular light dark pattern of fringes, with equal intensity in each of the bright regions. However, as noted above, the calculation did not take account of diffraction effects giving rise to varying diffraction efficiency. For the diffractive scenario under consideration, the diffraction efficiency will have a bell shaped curve that would limit the energy distribution in the diffracted beams. Therefore, applying such estimated diffraction efficiency curve to the result of FIG. 8A would lead to a distribution more closely resembling FIG. 8B, where the intensity of side lobes gradually decreases.

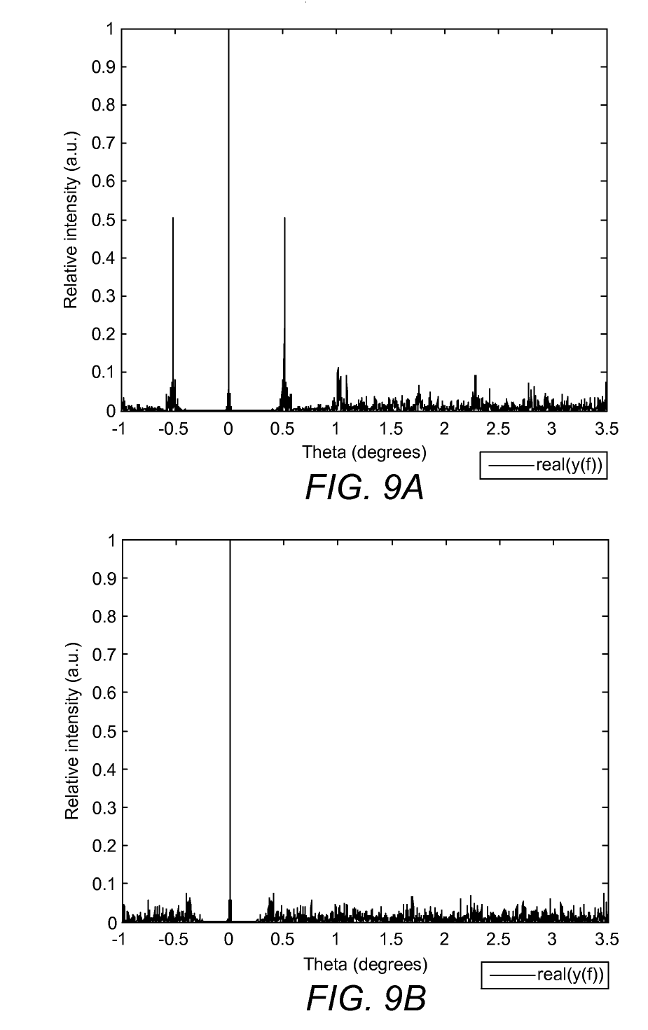

Now, recalculating the interference profile of FIG. 8A, but with emitter spacing randomised by up to +20 microns, it can clearly be seen in FIG. 9A that the intensity of the side lobes (either side of 0 degrees) is greatly reduced. If this randomised spacing is increased further, with a randomisation of up to +50 microns, so that the spacing of each prism from an adjacent prism can take any value from 51 microns to 101 microns, then the intensity in the side fringes either side of 0 degrees almost reduces to zero, as shown in FIG. 9B. This suggests that by applying randomised prism spacing to the Fresnel prism in the IOL, the diffraction effects can be greatly reduced, if not eliminated altogether.

A periodic and randomised structure with surface profile similar to FIG. 1 was cut into PMMA in a basic initial test. Both optics where illuminated with a laser and the resulting output light was imaged on a white screen. In both cases two distinct spots were observed as expected, but there was also noticeable scattered light in both cases. Although it was hard to perceive a dear difference due to the small number of grooves illuminated by the laser beam and the quality of the respective structures, the regular spacing optic scatter appeared to contain more structure, which was indicative of interference and diffractive effects.

The next step was to have a high-quality prism surface machined in PMMA using a fly-cutter arrangement. The manufacturing was a two-stage process. First the curved lens surface was machined, and the medical grade PMMA part was re-blocked (in a standard wax filled insert). The second side was then profiled to leave a raised central diameter into which the prism structure could be machined. The PMMA parts, still held in the blanks were then transferred for prism machining.

As the earlier prism lenses were compression moulded, two lens designs were machined from PMMA using a fly-cutter arrangement. One design comprised regular prism spacing_{;} as with the compression moulded lenses. This was produced to allow a comparison between the two different manufacturing processes and to ensure the machined regular spacing prism lens exhibited the same optical effects as the tested moulded lenses. The second design incorporated a randomised prism spacing, with spacing varying by an amount of 51 μm+(0 μm to 50 μm), i.e. the prism spacing ΔX varied in the range 51 μm≦ΔX≦101 μm. Tables 2A and 2B list the actual prism spacing Xn used for prisms X1 to X100 in the array. The spacing and resulting interference pattern side-lobe intensities were then calculated using the model simulation.

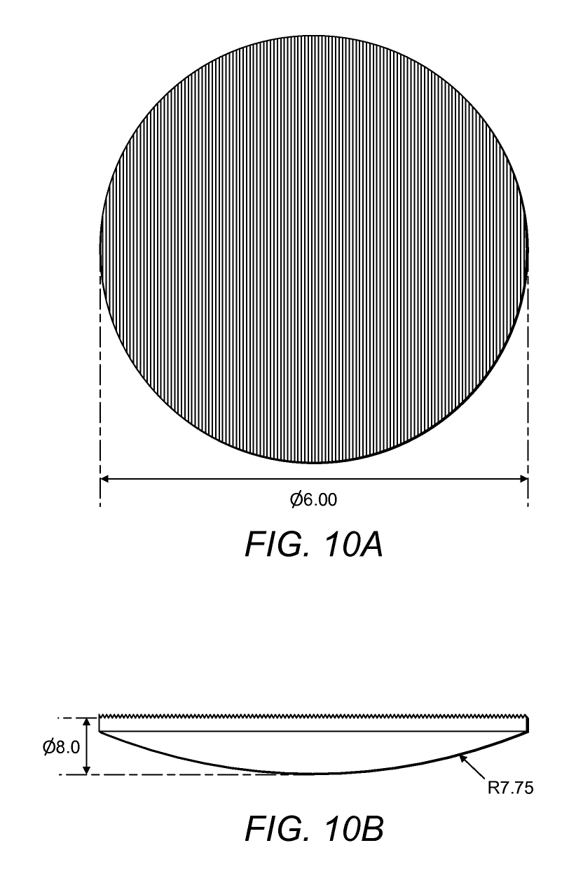

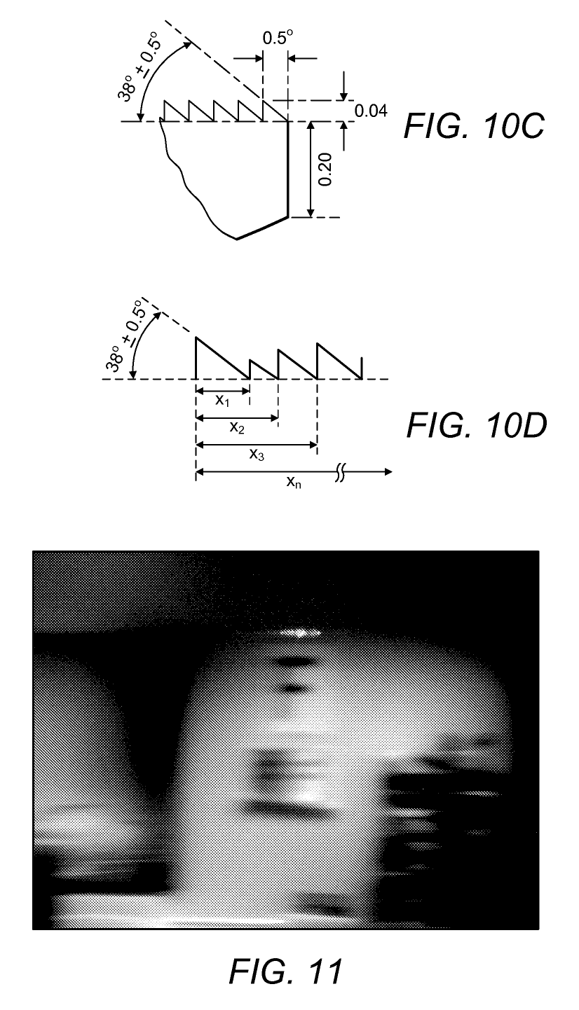

Due to the degrees of freedom available for automated adjustment on the lathe/fly-cutter, the depth of the cut remained constant. Therefore, as the prism spacing varied so the prism height varied. That is to say, the base of each prism was located at the same height, and so the apex heights varied with pitch. An example of the resulting Fresnel prism lens is illustrated in FIGS. 10A to 10D. It should be noted that the dimensions specified in these figures are merely illustrative of a particular configuration and could take other suitable values. FIGS. 10A and 10B, respectively, show a plan view and a side view of the Fresnel prism fabricated. FIG. 10C shows an expanded version of the Detail A from FIG. 10B, illustrating a prism array having uniform prism elements and pitch. In contrast, FIG. 10D Illustrates a section of the same prism array, but having a randomised pitch in accordance with an embodiment of the invention. The pitch Xₙ varied in the manner listed in Table 2A and 2B, and the angle of the prism facets was set at 38.0±0.5°, as indicated.

Once again, the lenses were tested using the optical bench arrangement described with reference to FIG. 4. Overall imaging quality was investigated using test targets (i.e. a letter F) and diffractive effects were investigated with monochromatic light. The chromatic dispersion of the optic was again investigated using a narrow band filter to limit the range of wavelengths passing through the optic.

FIG. 11 shows the resultant image obtained from a Fresnel prism IOL with the randomised prism spacing listed in Table 2. Although improved, the image quality was not improved by quite as much as expected, and so the model was revisited. On reviewing the model it became apparent that the interference intensity was calculated using all of the emitters. The effect of combining the effect due to all the emitter waves might result in destructive interference, whereas singling out the central 20 emitters, for example, might still give constructive interference, which is masked by the increased total illuminance when more emitters are used. Therefore, using the previous theoretical model, but considering the prism surfaces within the central 3 mm diameter zone and reviewing the interference effect, noticeable structure was indeed apparent. The model was therefore improved to incorporate an additional calculation for the emitters in the central 3 mm diameter region.

The simulation was then run repeatedly whilst monitoring the interference for both the entire surface and just the central 3 mm diameter. An additional important point is that the number of prisms (emitters) is tuned for the model, such that the total distance covered by the prisms matches that of the area of the optic onto which they will be machined. This ensured that the central 3 mm of the model matches the ‘real lens’ central 3 mm. FIGS. 12A and 12B and FIGS. 13A and 13B show the simulation results from this investigation and demonstrate the requirement for particular attention to be paid to the prism spacing over the central 3 mm diameter region.

In FIG. 12B there are clear constructive interference peaks visible at around 0.2 and 0.5 degrees that are not so apparent in the total surface plot shown in FIG. 12A. Therefore, as indicated above, the calculation was repeated using the same method, but concentrating on the randomisation in the central 3 mm zone. The results are shown in FIGS. 13A and 13B. As can be seen from FIG. 13B, the central 3 mm region exhibited greater ‘randomisation’, which removed any noticeable interference peaks, as compared to the results shown in FIG. 12B.

From the work described above, and as might be expected, the observed diffraction effect was lower for the larger prisms with larger associated randomised spacing. Therefore, in order to improve the performance of the Fresnel prism IOL of the present invention, the next step was to investigate a 21 D prism lens design with a 130 micron prism pitch and up to 130 microns of pitch randomisation. The exact prism pitch used for adjacent prisms X1-X40 is given in Tables 3A-3D. Furthermore, in this improved design, a toric lens (−5.5 D) was also placed just in front of the prism lens to provide for additional correction and remove the effect of the additional focusing power that is introduced by the prism surface operating in a converging beam. The −5.5 D was aligned to act in the same plane as the prism deviation. For the final IOL, the toric surface would be included in the IOL optic, such that the front surface will be 21 D parallel to prism rulings and 15.5 D perpendicular to prism rulings.

FIG. 14A illustrates an optical ray tracing 140 of this design though the complete simulated eye with the above IOL using Zemax tracing software. In the simulation, the cornea was 7.8 mm (k=−0.5) anterior (shown at 141) and 6.7 mm (k=−0.3) posterior (shown at 142). The simulation was based on an IOL 143 made of PMMA material with refractive index n=1.4915. The toric anterior surface (shown at 144) had radii of curvature R1=7.4 mm and R2=10.0 mm, Ct0.80 mm, and the posterior surface (shown at 145) was plano with a Fresnel prism structure having the spacing detailed in Table 3. This design resulted in a 21 D lens, with an effective 15.5 D (−5.5 D) parallel to the deviation plane, to account for the extra focusing power of the prism surface in the converging rays.

FIG. 14B shows the ray-traced image of a letter “F” though the system of FIG. 14A using the Zemax software, whilst FIGS. 14C and 14D show the associated spot diagrams. The actual observed image of the letter F through the improved lens using the optical bench model eye test equipment is shown in FIG. 15C. For comparison, FIG. 15A shows the image produced by a PMMA 26.5 D standard spherical lens and FIG. 15B shows the image produced by a 21 D Fresnel prism lens with machined regular spacing.

As is apparent, whilst the image quality produced by the randomised Fresnel prism array IOL with toric anterior surface may not be quite as good as produced by a conventional spherical lens, it is far superior to the regularly-spaced Fresnel prism array IOL. Moreover, as can be seen by comparing to FIG. 11, it is superior to the previously-described randomised Fresnel prism array IOL having smaller prism size and pitch and no toric anterior surface. Although some improvement in the image quality is attributable to the toric surface, the majority of the improvement (over FIG. 11) is due to the lager prism spacing and randomisation.

Thus, as has been demonstrated, in an IOL according to the present invention with randomised prism spacing, the image quality is greatly improved, as compared to the known Fresnel prism IOL design. When a toric lens or lens surface is added, the image quality is improved further, and the astigmatic aberration almost eliminated. In some designs of a Fresnel prism IOL according to the invention, such toric surface can be supplemented or replaced by suitable variation in the facet angle of the prisms in the array, such that astigmatism that would otherwise be introduced by the prism elements is compensated for.

The improved imaging quality of a Fresnel prism IOL according to the present invention makes such a lens a very promising candidate for the surgical treatment of macular degeneration conditions, including age-related macular degeneration (ARMD). Careful design of the lens should enable a customised lens to be produced for the treatment of a patient with such a condition by enabling the point of image formation to be deviated to a healthy part of the retina, whilst retaining a high quality of image formation at the deviated position.

TABLE 2A

| Prism | Position | |
|---|---|---|
| number | X (μm) | ΔX (μm) |
| | 0 | |
| X1 | 51 | 51 |
| X2 | 141 | 90 |
| X3 | 208 | 67 |
| X4 | 282 | 74 |
| X5 | 371 | 89 |
| X6 | 427 | 56 |
| X7 | 484 | 56 |
| X8 | 548 | 64 |
| X9 | 625 | 77 |
| X10 | 725 | 100 |
| X11 | 811 | 87 |
| X12 | 878 | 67 |
| X13 | 944 | 66 |
| X14 | 1037 | 94 |
| X15 | 1134 | 97 |
| X16 | 1217 | 83 |
| X17 | 1280 | 64 |
| X18 | 1336 | 55 |
| X19 | 1429 | 93 |
| X20 | 1509 | 80 |
| X21 | 1607 | 98 |
| X22 | 1661 | 54 |
| X23 | 1742 | 80 |
| X24 | 1807 | 65 |
| X25 | 1899 | 92 |

TABLE 2B

| Prism | Position | |
|---|---|---|
| number | X (μm) | ΔX (μm) |
| X26 | 1960 | 61 |
| X27 | 2033 | 73 |
| X28 | 2104 | 71 |
| X29 | 2196 | 92 |
| X30 | 2281 | 85 |
| X31 | 2342 | 61 |
| X32 | 2409 | 67 |
| X33 | 2467 | 58 |
| X34 | 2551 | 85 |
| X35 | 2631 | 80 |
| X36 | 2690 | 59 |
| X37 | 2749 | 58 |
| X38 | 2824 | 75 |
| X39 | 2920 | 96 |
| X40 | 2999 | 79 |
| X41 | 3051 | 53 |
| X42 | 3105 | 54 |
| X43 | 3196 | 91 |
| X44 | 3270 | 74 |
| X45 | 3340 | 70 |
| X46 | 3430 | 90 |
| X47 | 3500 | 69 |
| X48 | 3577 | 78 |
| X49 | 3664 | 87 |
| X50 | 3758 | 95 |

TABLE 2C

| Prism | Position | |
|---|---|---|
| number | X (μm) | ΔX (μm) |
| X51 | 3826 | 67 |
| X52 | 3909 | 84 |
| X53 | 4009 | 100 |
| X54 | 4064 | 55 |
| X55 | 4144 | 80 |
| X56 | 4216 | 72 |
| X57 | 4282 | 66 |
| X58 | 4347 | 64 |
| X59 | 4436 | 89 |
| X60 | 4536 | 101 |
| X61 | 4597 | 60 |
| X62 | 4687 | 90 |
| X63 | 4747 | 61 |
| X64 | 4848 | 101 |
| X65 | 4939 | 91 |
| X66 | 5011 | 72 |
| X67 | 5099 | 87 |
| X68 | 5175 | 76 |
| X69 | 5266 | 91 |
| X70 | 5335 | 69 |
| X71 | 5390 | 55 |
| X72 | 5470 | 81 |
| X73 | 5567 | 97 |
| X74 | 5627 | 61 |
| X75 | 5700 | 73 |

TABLE 2D

| Prism | Position | |
|---|---|---|
| number | X (μm) | ΔX (μm) |
| X76 | 5789 | 88 |
| X77 | 5841 | 53 |
| X78 | 5940 | 98 |
| X79 | 6029 | 89 |
| X80 | 6108 | 79 |
| X81 | 6168 | 60 |
| X82 | 6244 | 76 |
| X83 | 6321 | 77 |
| X84 | 6422 | 101 |
| X85 | 6515 | 94 |
| X86 | 6614 | 99 |
| X87 | 6699 | 85 |
| X88 | 6771 | 71 |
| X89 | 6868 | 98 |
| X90 | 6943 | 75 |
| X91 | 7006 | 63 |
| X92 | 7077 | 71 |
| X93 | 7163 | 86 |
| X94 | 7242 | 79 |
| X95 | 7331 | 89 |
| X96 | 7432 | 101 |
| X97 | 7531 | 99 |
| X98 | 7608 | 78 |
| X99 | 7708 | 99 |
| X100 | 7764 | 57 |

TABLE 3A

| Prism | Position | ΔX |
|---|---|---|
| number | X (μm) | (μm) |
| | 0 | |
| X1 | 130 | 130 |
| X2 | 306 | 176 |
| X3 | 520 | 214 |
| X4 | 771 | 251 |
| X5 | 913 | 142 |
| X6 | 1139 | 226 |
| X7 | 1276 | 137 |
| X8 | 1505 | 228 |
| X9 | 1695 | 190 |
| X10 | 1831 | 136 |
| X11 | 2070 | 239 |
| X12 | 2222 | 151 |
| X13 | 2367 | 145 |
| X14 | 2532 | 165 |
| X15 | 2703 | 171 |
| X16 | 2912 | 209 |
| X17 | 3130 | 218 |
| X18 | 3388 | 258 |
| X19 | 3647 | 259 |
| X20 | 3876 | 228 |

TABLE 3B

| Prism | Position | ΔX |
|---|---|---|
| number | X (μm) | (μm) |
| X21 | 4041 | 166 |
| X22 | 4295 | 254 |
| X23 | 4479 | 183 |
| X24 | 4637 | 158 |
| X25 | 4849 | 212 |
| X26 | 4981 | 132 |
| X27 | 5116 | 136 |
| X28 | 5270 | 153 |
| X29 | 5426 | 156 |
| X30 | 5649 | 224 |
| X31 | 5837 | 188 |
| X32 | 6077 | 240 |
| X33 | 6257 | 181 |
| X34 | 6496 | 239 |
| X35 | 6724 | 227 |
| X36 | 6930 | 206 |
| X37 | 7080 | 151 |
| X38 | 7279 | 199 |
| X39 | 7469 | 190 |
| X40 | 7649 | 179 |

1. An intraocular lens having an optical axis, the lens comprising, as one face thereof, a Fresnel prism comprising an array of elongate prism elements which are parallel to one another along their length, each prism element having an elongate facet which is oriented such that a perpendicular to the facet is at an angle to the optical axis,
wherein the array of prism elements is configured to deviate light incident thereon to an off-axis position lying in a plane defined by the optical axis and the perpendicular to any of the angled facets,
and wherein one or more of the pitch and the size of prism elements is non-uniform across the array and is selected to reduce a diffraction grating effect associated with the array of prism elements, whereby light incident on the lens is preferentially directed into the zero order diffraction direction and chromatic angular dispersion is reduced.

2. A lens according to claim 1, wherein one or more of the pitch and the size of prism elements in the array has been randomised to reduce the diffraction grating effect.

3. A lens according to claim 2, wherein the randomisation is different in a region of the array proximate the optical axis as compared to a region distal the optical axis.

4. A lens according to claim 1, wherein the pitch of the prism elements in the array is in the range 50 μm to 500 μm.

5. A lens according to claim 1, wherein the pitch of the prism elements in the array varies by an amount in the range 0 μm to 50 μm.

6. A lens according to claim 1, wherein the pitch of the prism elements in the array varies by an amount in the range 0 μm to 130 μm.

7. A lens according to claim 1, wherein a facet angle of prism elements is non-uniform across the array and is selected to compensate for astigmatism that would otherwise result from the presence of the Fresnel prism.

8. A lens according to claim 7, wherein the facet angles vary monotonically across at least a portion of the array to compensate for the astigmatism.

9. An intraocular lens having an optical axis, the lens comprising, as one face thereof, a Fresnel prism comprising an array of elongate prism elements which are parallel to one another along their length, each prism element having an elongate facet which is oriented such that a perpendicular to the facet is at an angle to the optical axis,
wherein the array of prism elements is configured to deviate light incident thereon to an off-axis position lying in a plane defined by the optical axis and the perpendicular to any of the angled facets,
and wherein the angle of the prism element facets is non-uniform across the array and is selected to compensate for astigmatism that would otherwise result from the presence of the Fresnel prism.

10. A lens according to claim 9, wherein the facet angles vary monotonically across at least a portion of the array to compensate for the astigmatism.

11. A lens according to claim 1, wherein the angle of the facets is in the range 37.5 to 38.5 degrees.

12. A lens according to claim 1, wherein the prism elements are formed on a planar surface.

13. A lens according to claim 1, wherein the prism elements are formed on a non-planar surface.

14. A lens according to claim 1, further comprising a material covering said one face, thereby providing a smooth surface.

15. A lens according to claim 1, wherein another face of the lens comprises a tonic shape to compensate for astigmatism introduced by the Fresnel prism face.

16. A lens according to claim 1, wherein the lens is configured for use with the Fresnel prism on the anterior surface.

17. A lens according to claim 1, wherein the lens further includes one or more haptics attached to the lens at its perimeter.

18. A combination of an intraocular lens according to claim 1, and a second intraocular lens.

19. A combination according to claim 18, wherein the second lens has a toric shape to compensate for astigmatism in the lens combination.

20. A method for the treatment of a macular condition requiring a change of focused image position, which comprises replacing a patient's crystalline lens by a lens according to claim 1.

21. A method for the treatment of a macular condition requiring a change of focused image position, which comprises implanting into a patient's eye a lens according to claim 1 in order to supplement the patient's crystalline lens or an existing intraocular lens or lens combination.

22. A method according to claim 20, wherein the macular condition is age-related macular degeneration.

23. A lens according to claim 9, wherein the angle of the facets is in the range 37.5 to 38.5 degrees.

24. A method for the treatment of a macular condition requiring a change of focused image position, which comprises implanting into a patient's eye a lens according to claim 9.
